(19) United States
(12) Patent Application Publication
(10) Pub. No.: US 2009/0098641 A1
(43) Pub. Date: Apr. 16, 2009

(54) Incubator humidity control

(76) Inventor: Paul Simon Grant, Cambridge (GB)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 12/232,543

(22) Filed: Sep. 18, 2008

(30) Foreign Application Priority Data
Sep. 20, 2007 (EP) 07116873.6

(51) Int. Cl. C12M 1/36

(52) U.S. Cl. 435/286.1

(57) ABSTRACT

An incubator 1 for cell culture, or biological or chemical samples is provided, the incubator having disposed therein a humidity sensor 28 and a humidifier 2 having an atomiser including a spray nozzle 21. A compressed air supply line 25 and a liquid supply line 26 are connected to the spray nozzle. A control system 30 allows the required humidity level to be set, and is connected to the humidity sensor 28 to receive a signal indicative of the humidity within the incubator, and is also connected to the humidifier 2 to operate the humidifier to maintain the humidity within the incubator at the set level.

### CROSS-REFERENCE TO RELATED PATENT APPLICATION

This application claims the benefit under 35 U.S.C. §119(a) of European Patent Application No. 07116873.6, filed Sep. 20, 2007, in the European Patent Office, the entire disclosure of which is hereby incorporated by reference.

### BACKGROUND OF THE INVENTION

1. Field of the Invention

The present invention relates to incubators for cell culture, or biological or chemical samples, for example of the type shown in EP-A-0569214 or EP-A-1657552.

More particularly, the invention is concerned with maintaining a desired level of humidity in such incubators.

2. Description of the Related Art

It is well known that, in order to allow certain cell culture, or biological or chemical processes to function satisfactorily the samples held within vessels in such an incubator need to be maintained at both a desired temperature or range of temperatures and a suitable humidity or range of humidity. In particular, when using SBS Format or similar microtitre plates with loose fitting lids, because greater evaporation otherwise occurs at the wells adjacent the edges of the plates, maintaining a relatively high humidity helps to avoid lack of uniformity in evaporation.

While it has been proposed to provide incubators with humidifiers using either heated water sources or with ultrasonic atomisers, it is desirable to avoid adding heat through the humidifier because this will then create undesirable hot spots within the incubator. Ultrasonic units in particular, provide undesirable heat sources. A further problem with prior humidifying systems is that. from normal lab conditions, conventional humidity devices may take an hour or more to restore high humidity (>90%), which is undesirable if there is continual access.

### SUMMARY OF THE INVENTION

Exemplary embodiments of the present invention address at least the above problems and/or disadvantages and provide at least the advantages described below.

According to an exemplary embodiment of the present invention therefore there is provided an incubator for cell culture, or biological or chemical samples, the incubator having disposed therein:

a humidity sensor;

a humidifier having an atomiser including a spray nozzle, and a compressed air supply line and a liquid supply line to the spray nozzle; and

a control system in which the required humidity level can be set, connected to the humidity sensor to receive a signal indicative of the humidity within the incubator, and connected to the humidifier to operate the humidifier to maintain the humidity within the incubator at the set level.

### BRIEF DESCRIPTION OF THE DRAWINGS

The above and other objects, features, and advantages of certain embodiments of the present invention will be more apparent from the following description taken in conjunction with the accompanying drawings, in which:

FIGS. 1 and 2 show an incubator with plural supports for multiple racks of sample flasks constructed in accordance with an exemplary embodiment of the present invention;

FIG. 3 illustrates the components of a humidifying system associated with the incubator in accordance with an exemplary embodiment of the present invention; and

FIG. 4 illustrates a humidifier constructed in accordance with an exemplary embodiment of the present invention.

Throughout the drawings, the same drawing reference numerals will be understood to refer to the same elements, features, and structures.

### DETAILED DESCRIPTION OF EXEMPLARY EMBODIMENTS OF THE PRESENT INVENTION

The matters defined in the description such as a detailed construction and elements are provided to assist in a comprehensive understanding of the embodiments of the invention and are merely exemplary. Accordingly, those of ordinary skill in the art will recognize that various changes and modifications of the embodiments described herein can be made without departing from the scope and spirit of the invention. Also, descriptions of well-known functions and constructions are omitted for clarity and conciseness.

According to an exemplary embodiment of the present invention, therefore, there is provided an incubator for cell culture, or biological or chemical samples, the incubator having disposed therein:

a humidity sensor;

a humidifier having an atomiser including a spray nozzle, and a compressed air supply line and a liquid supply line to the spray nozzle; and

a control system in which the required humidity level can be set, connected to the humidity sensor to receive a signal indicative of the humidity within the incubator, and connected to the humidifier to operate the humidifier to maintain the humidity within the incubator at the set level.

Preferably, the humidifier includes a venturi.

Suitable humidifiers are manufactured by Delavan® and utilise compressed air to draw water into the nozzle over the venturi and to atomise it.

Preferably, the humidity sensor includes a PID controller and this is connected to a compressed air solenoid valve to determine the amount of compressed air and hence atomised water delivered into the incubator.

The exemplary system of the present invention also provides a very fast recovery speed after humidity has dropped when the door has been opened by an operator or by the robotics outside the incubator.

If desired, the compressed air may be filtered, for example using a standard SMC filter to filter out particles such as undesirable bacteria, having a diameter greater than 0.1 μm. The liquid (water) supply to the humidifier may also include a solenoid valve, depending on the head of water in the supply and may include suitable filters also.

One example of an incubator according to the present invention will now be described with reference to the accompanying drawings in which:

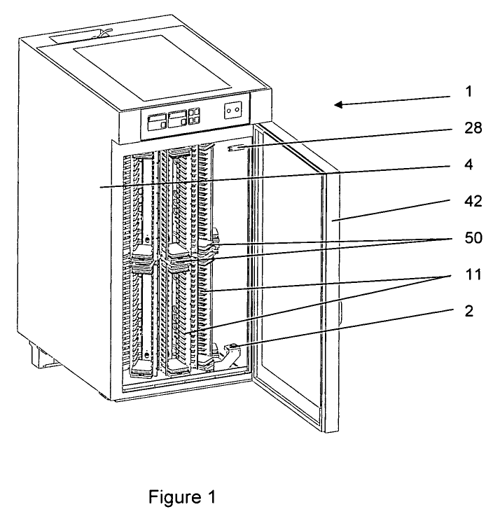

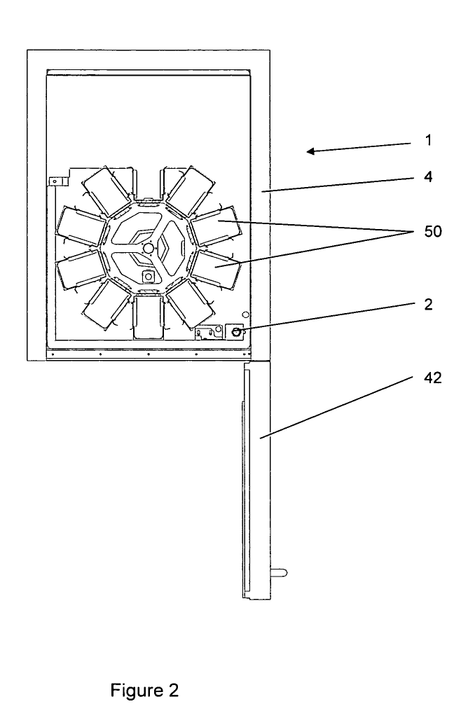

FIGS. 1 and 2 show an incubator in accordance with an exemplary embodiment of the present invention having plural supports for multiple racks of sample flasks;

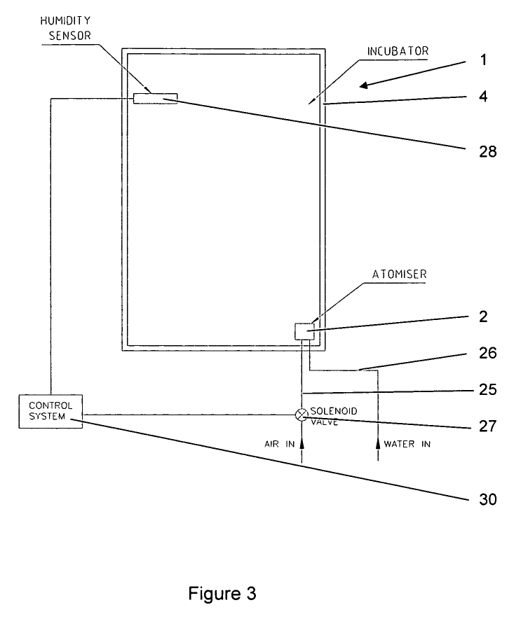

FIG. 3 illustrates the components of a humidifying system associated with the incubator; and

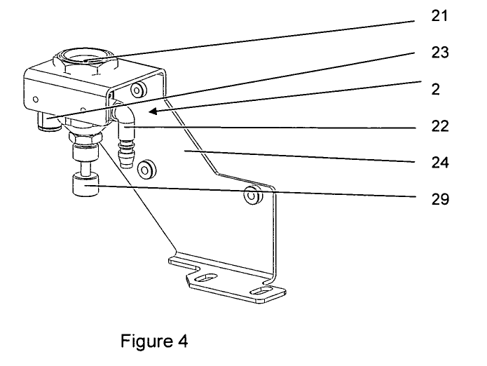

FIG. 4 illustrates the humidifier in more detail.

FIG. 1A is a perspective view of an incubator and FIG. 2 is a plan view of the incubator. The incubator 1 shown in the drawing includes a housing 4 in order to enable a controlled environment to be maintained and adjusted. The incubator housing 4 may include (not shown) a slidable access panel or door 41 through which a transporter in the form of a robotic arm with a gripper unit may be extended to load/unload culture vessels such as SBS format microtitre plates 50. The housing has a glass paneled operator access door 42.

The incubator 1 includes a plurality of microtitre plate ‘hotels’ 11 which are disposed so as to be rotatable for access, about a vertical axis generally centrally disposed within the incubator, the hotels 11 support the plates 50 in columns around the common axis to provide what is, effectively a carousel.

As the remaining components of the incubator shown in FIGS. 1 and 2 are not related to the present invention they will not be further described.

As can be seen in FIGS. 1 to 3, at the bottom of the incubator 1, there is positioned a humidifier 2 which includes a spray nozzle 21, a compressed air supply line connector 23 connecting a compressed air line 25, and a liquid supply line connector 22 connecting a water feed line 26 to the spray nozzle. The humidifier is mounted to the housing 4 via a bracket 24. An externally positioned control system 30 (see FIG. 3) allows the required humidity level to be set by controlling a solenoid valve 27 in the compressed air line 25 which feeds the humidifier 2. The control system 30 is also connected to a humidity sensor 28, positioned inside and near the top of the incubator housing 4, in order to receive a signal indicating the humidity within the incubator 1. The control system 30 is connected to operate the humidifier to maintain the humidity within the incubator at a level set within the control system.

The humidifier shown in the drawings can be, for example, a Delavan® AL air atomising series spray nozzle which uses the energy of the pressurised (compressed) air to atomise the water and this allows the water to be fed under lower pressure and still achieve fine atomisation. It includes an adjusting valve, the control knob 29 of which is seen in FIG. 4

The combination of a humidifier 2 of this type with an appropriate humidity sensor 28 and control system 30 allows a simple and cost effective means of producing the desired humidity environment in the incubator 1 without introduction of any significant heat.

The illustrative system of the exemplary embodiment of the present invention can achieve 95% RH in a matter of a few minutes.

While the invention has been shown and described with reference to certain embodiments thereof, it will be understood by those skilled in the art that various changes in form and details may be made therein without departing from the spirit and scope of the invention as defined by the appended claims.

What is claimed is:

1. An incubator for cell culture, or biological or chemical samples, the incubator having disposed therein:
a humidity sensor;
a humidifier having an atomiser including a spray nozzle, and a compressed air supply line and a liquid supply line to the spray nozzle; and
a control system in which the required humidity level can be set, connected to the humidity sensor to receive a signal indicative of the humidity within the incubator, and connected to the humidifier to operate the humidifier to maintain the humidity within the incubator at the set level.

2. An incubator according to claim 1, wherein the incubator has a venturi atomiser.

3. An incubator according to claim 1 or claim 2 further including a filter in the compressed air supply line.

4. An incubator according to claim 1 or claim 2, wherein the liquid supply line includes a filter disposed therein.
